SEQUENCE LISTING (4 sequences)

SEQ ID NO: 1 - RNA, 21 nt, Artificial
  synthetically constructed siRNA against Parp-1
gaaaguguguucaacuaauuu

SEQ ID NO: 2 - RNA, 19 nt, Artificial
  synthetically constructed siRNA against Parp-1
gcaacaacuggaacagauu

SEQ ID NO: 3 - RNA, 21 nt, Artificial
  synthetically constructed siRNA against XRCC3
ggaccugaaucccagaauuuu

SEQ ID NO: 4 - DNA, 21 bp, Artificial
  synthetically constructed siRNA against luciferase
cguacgcggaauacuucgatt

(19) United States
(12) Patent Application Publication
(10) Pub. No.: US 2012/0022026 A1
(43) Pub. Date: Jan. 26, 2012

(54) METHOD OF TREATING CANCER

(75) Inventors: Przemyslaw Krawczyk, Amsterdam (NL); Jacob A. Aten, Amsterdam (NL); Roland Kanaar, Rotterdam (NL); Jeroen Essers, Rotterdam (NL)

(73) Assignees: ERASMUS UNIVERSITY MEDICAL CENTER ROTTERDAM, Rotterdam (NL); ACADEMISCH MEDISCH CENTRU BIJ DE UNIVERSITEIT VAN AMSTERDAM, Amsterdam (NL)

(21) Appl. No.: 13/144,079

(22) PCT Filed: Jan. 13, 2010
(86) PCT No.: PCT/NL2010/050016

§ 371 Date: Sep. 30, 2011

(30) Foreign Application Priority Data
Jan. 13, 2009 (NL) PCT/NL2009/050007

(51) Int. Cl. A61K 31/65; A61K 31/351; A61K 31/435; A61K 31/517; A61P 35/00; A61K 31/495; A61K 31/4184; A61K 31/40; A61K 31/502; C12N 5/0735; A61K 31/47; A61K 31/395

(52) U.S. Cl. 514/152; 514/312; 514/307; 514/460; 514/298; 514/266.1; 514/183; 514/252.12; 514/394; 514/427; 514/248; 435/375

(57) ABSTRACT

The present invention relates to a method of treating or preventing hyperproliferative disease in a body tissue of a subject, comprising the steps of administering to a subject in need thereof a therapeutically effective amount of an agent that induces double strand breaks in the DNA of the hyperproliferative cells of said body tissue; and subjecting the hyperproliferative cells of said body tissue prior to, simultaneously with or subsequent to step a) to hyperthermia to thereby induce in said cells the degradation, inhibition and/or inactivation of BRCA2.

### TECHNICAL FIELD

The present invention relates to methods of treating or preventing hyperproliferative disease. In particular, the present invention relates to compounds and compositions for the treatment of cancer. The invention further relates to a method for inhibiting homologous recombination in cells, to a method of killing cells, and to the use of known anti-cancer drugs in new therapeutic applications, in particular for the manufacture of medicaments for combination anti-cancer therapy.

### BACKGROUND OF THE INVENTION

Many currently applied anti-cancer strategies are based on cytotoxicity of DNA double-strand breaks (DSBs) induced by ionizing radiation or, indirectly, by chemical agents. However, efficient DSB repair mechanisms protect cells from the genotoxic effects of DSBs, reducing the effectiveness of the treatment. Two major DSB repair pathways have been described in mammalian cells: homologous recombination (HR) and non-homologous end joining (NHEJ). HR utilizes intact homologous DNA sequences—usually the sister chromatid in post-replicative chromatin—to faithfully restore DNA breaks. Major HR factors include the DNA strand exchange protein RAD51 and the recombination mediators BRCA2, XRCC3 and RAD54. NHEJ operates throughout the entire cell cycle and does not require a DNA template. Core NHEJ functions are performed by the Ku70/80 heterodimer, DNA-PKCS, DNA LigIV and XRCC4. Inhibition of DSB repair processes potentiates the cytotoxic effects of induced DSBs. Methods for disrupting DNA DSB repair pathways are rapidly gaining importance in anti-cancer therapy. However, inhibiting DSB repair pathways has adverse effects on normal cells, which need to cope with DSBs arising during metabolic activities. Hence, specificity for cancer cells is needed.

Hyperthermia (also called thermal therapy or thermotherapy) is a type of cancer treatment in which the affected body tissue is exposed to high temperatures (up to 45 or 46° C.). Research has shown that high temperatures can damage and kill cancer cells, usually with minimal injury to non-heated, surrounding normal tissue. By killing cancer cells and damaging proteins and structures within cells, hyperthermia may reduce tumor volume. Additionally, hyperthermia is known to make cancer cells more sensitive to other forms of cancer therapy, such as radiation therapy and chemotherapy, and to sensitize cancer cells to certain anticancer drugs. The treatment is beneficial in treating many types of cancer, including sarcoma, melanoma, and cancers of the head and neck, brain, lung, esophagus, breast, bladder, rectum, liver, appendix, cervix, and peritoneal lining (mesothelioma). Some malignant cell masses having poorer heat dissipation characteristics than normal tissue, presumably due to abnormally low blood circulation, are subject to preferential hyperthermia treatment. As a result, the temperature of such malignant cell masses is often raised to substantially higher values than that of surrounding healthy cells even when both types of cells are heated simultaneously. This characteristic enables hyperthermia treatment of malignancies with a comparable temperature sensitivity to normal cells, and also permits much shorter hyperthermia treatment times, allowing treatment of malignancies in thermally sensitive tissues. Some malignant growths have a relatively narrow hyperthermia treatment temperature range. It is generally believed that below temperatures of about 41.5° C., thermal destruction of these malignancies does not occur, and may even stimulate tumor growth. In contrast, at temperatures above a range of about 43° C. to 45° C. even normal cells are thermally damaged when duration of exposure is prolonged. But if large or critical parts of a human body are heated into, or above, the 43° C. to 45° C. range for even relatively short durations, serious permanent injury or death is possible. Hence, there is a need to be able to calibrate the therapeutic dosage of hyperthermia, and it is an aim of the present invention to achieve that.

Hyperthermia is one of the oldest clinically applied agents enhancing the effectiveness of various anti-cancer therapies, but its exact mode of action remains elusive. Hence, the optimal treatment conditions for hyperthermia are unknown in many instances. Against this background there remains a need for improved methods of cancer treatment wherein hyperthermia is one of the therapeutic means.

### SUMMARY OF THE INVENTION

The inventors have now found that HR in mammalian cells can be inhibited by subjecting those cells to mild hyperthermia (an elevation in the temperature to between about 41.0-43.0° C.). The inventors have come to their finding by observing that in mammalian cells hyperthermia results in a decrease in the level of BRCA2. The effect of hyperthermia on BRCA2 and/or HR has hitherto not been reported.

It is known that BRCA2 is involved in recombinational repair of DSBs and mutations in this gene are known to be associated with an increased risk of hereditary breast cancer. It is further known that PARP (poly(ADP-ribose) polymerase) inhibitors, substances that result in the induction of DSBs in DNA, are effective in killing such BRCA2-defective breast cancer cells due to their flawed DSB repair. Hence, PARP inhibitors are indicated as a medicament for the treatment of BRCA2-defective breast cancer. Thus, although the relationship between BRCA2 and PARP is well established in the art, the interaction with hyperthermia has not previously been addressed.

The present inventors have found that hyperthermia induces degradation or inhibition of BRCA2 in mammalian cells. Based thereon, it was predicted (and indeed found) that hyperthermia of mammalian cells can be used to inhibit BRCA2-mediated repair of DSBs. This enables the extension of the use of PARP inhibitors in anti-cancer treatment of non-BRCA2/BRCA1-defective tumor types, i.e. in cells lacking a defect in HR repair, because such cells can be made HR-deficient by hyperthermia.

The present invention provides a method for treating cancer comprising administering to a subject in need of such treatment an agent that induces DSBs in DNA while simultaneously inducing degradation or inhibition of BRCA2 and inhibition of HR in tumor cells by hyperthermia. The advantage of this method is that it allows for optimization of currently available treatments, in particular the monitoring and optimization of the hyperthermia treatment component relative to the DSB induction. For instance, it is expected that dosage regimes for ionizing radiation treatment can be lowered as a result of this. More importantly, during DSB-inducing medication, hyperthermia in combination with monitoring of BRCA2 levels can assist in maintaining the cells in their most sensitive state.

The present inventors found that the efficacy of the combination therapy described above was enhanced considerably by the simultaneous application of heat shock protein (HSP) inhibitors, in particular inhibitors of HSP-90. It was found that this particular embodiment resulted in a killing of up to 98% of BRCA2-proficient cells in vitro. In comparison, PARP-1 inhibitors alone killed 40% of the cells, and the combination of hyperthermia and PARP-1 inhibitors killed 76% of the cells. In a preferred embodiment, therefore, a method of the invention further comprises the step of administering to said subject a HSP inhibitor. As an example, the use of the HSP-90 inhibitor 17-DMAG in combination with both hyperthermia and the use of PARP-1 inhibitors is contemplated. Therefore, the use of PARP-1 inhibitors, hyperthermia and HSP inhibitors, constitutes a preferred embodiment of the present invention.

The discovery of the mechanism of hyperthermic cell sensitization allows for the provision of several new and inventive cell killing methods. These will find important utility in anti-cancer treatments. The cell killing methods are very suitably local cell killing methods, meaning that only a part of the body, preferably a malignant growth or tumor, is targeted so that the effects are restricted to the tumour volume.

In a broad aspect, the present invention provides a method for inhibiting HR in cells comprising inducing degradation or inhibition of BRCA2 in said cells by hyperthermia. The method thus entails subjecting the cells to hyperthermia to a level that effectively inhibits, degrades or inactivates BRCA2 in said cells. The resulting HR deficiency in said cells renders them sensitive to DSB inducing agents.

In another aspect, the present invention provides a method of killing cells, preferably a population of hyperproliferative tissue cells in a subject in need thereof, said method comprising the step of administering to said subject a therapeutically effective (DSB-inducing) amount of a DSB inducing agent preferably selected from agents that induce DSBs which are known to be repaired by BRCA2-assisted DSB repair pathways, thereby inducing DSBs in the DNA of said cells, said method further comprising the step of inducing inhibition of HR in said cells by hyperthermia. The steps may be performed subsequently to oneanother in any order, but are in certain embodiments preferably performed simultaneously.

In another aspect, the present invention provides the use of a PARP-inhibitor for the preparation of a medicament for killing cells or tumors or retarding the growth of cells or tumors within a subject in accordance with a treatment regimen involving (a) administering to the cell or tumor within the subject a therapeutically effective amount of the medicament comprising the PARP-inhibitor and (b) prior to, simultaneously with, or subsequently to inducing inhibition of HR in said cell or tumor within the subject by hyperthermia. Preferably the subject is a human. As indicated, the cells to be killed in aspects of the invention may be normal tissue cells, but are preferably hyperproliferative cells, eg. cancer cells.

In another aspect, the present invention provides a method for killing cells comprising inhibiting HR in said cells by subjecting said cells to hyperthermia to the extend that BRCA2 mediated DNA repair is inhibited and exposing said cells to a DSB-inducing agent.

In a preferred embodiment of aspects of the invention, the method of killing cells or tumors further comprises exposing said cells to a HSP inhibitor, preferably a HSP-90 inhibitor, more preferably 17-DMAG.

The above method of killing cells is preferably part of a method of treating cancer and, therefore, said cells are preferably malignant cells.

In aspects of the present invention said DSB-inducing agent is preferably a PARP inhibitor. Suitable PARP-inhibitors include PARP-1 and PARP-2 inhibitors, including, but not limited to:
5-aminoisoquinolinone (5-AIQ);
3-methyl-5-AIQ (IC50=0.23 μM);
3-aminobenzamide (3-ABA) (EC50=200 μM);
5-iodo-6-amino-1,2-benzopyrone (INH2BP);
3,4-dihydro-5[4-(1-piperindinyl)butoxy]-1(2H)-isoquinoline (DPQ) (IC50=40 nM);
1,5-dihydroxyisoquinoline (IQD) (IC50=390 nM);
aza-5[H]-phenanthridin-6-ones (aza-PHE);
4-amino-1,8-naphthalimide (4-ANI) (IC50=180 nM);
8-hydroxy-2-methylquinazoline-4-one (NU1025) (IC50=0.40 μM);
N-(6-oxo-5,6-dihydrophenanthridin-2-yl)-N,N-dimethylacetamide (PJ-34) (EC50=20 nM);
indeno-isoquinolinone (INO-1001) (Inotek);
5-chloro-2-[3-(4-phenyl-3,6-dihydro-1(2H)-pyridinyl)propyl]-4(3H)-quinazolinone (FR247304);
1-piperazineacetamide,4-[1-(6-amino-9H-purin-9-yl)-1-deoxy-6-D-ribofuranuron]-N-(2,3-dihydro-1H-isoindol-4-yl)-1-one (EB-47) (IC50=45 nM);
thieno[2,3-c]isoquinolin-5-one (TIQ-A) (IC50=450 nM);
2-Dimethylaminomethyl-4H-thieno [2,3-c]isoquinolin-5-one (DAM-TIQ-A);
4-hydroxyquinazoline;
nicotinamide;
minocycline (competitive inhibition Ki=13.8 nM for recombinant PARP-1 in a cell-free assay);
2-methyl-3,5,7,8-tetrahydrothiopyrano[4,3-d]pyrimidine-4-one (DR2313) (IC50=200 nM and 240 nM for PARP-1 and PARP-2, respectively);
3-(4-chlorophenyl)quinoxaline-5-carboxamide ((IC50=7 and 33 nM for PARP-2 and PARP-1 respectively);
benzamide;
N-(6-oxo-5,6-dihydrophenanthridin-2-yl)-2-(N,N-dimethylamino)acetamide (PJ34) (Inotek Corporation)
AG014699 (CAS No: 459868-92-9) and/or AG14361 (Pfizer)
2-[(R)-2-methylpyrrolidin-2-yl]-1H-benzimidazole-4-carboxamide (ABT-888) (Abbott)
4-[3-(4-cyclopropanecarbonylpiperazine-1-carbonyl)-4-fluorobenzyl]-2H-phthalazin-1-one (Olaparib; AZD2281; KU-0059436) (AstraZeneca)
BSI-401 (CAS No: 142404-10-2) and BSI-201 (4-iodo-3-nitrobenzamide; CAS No: 160003-66-7; Cancer Biol Ther. January 2009; 8(1):2-3) (Bipar)
CEP-8983 (WO 01/85686) or its prodrug (CEP-9722) (Cephalon)
GPI-21016 (WO 99/11645), GPI 16346 and GPI 18180, GPI 6150, GPI 18078; GPI 6000 (MGI Pharma)
2-aminothiazole analogues (in particular compounds 4-6 and 10 in W.-T. Zhang et al., J. Med. Chem., 2009, 52 (3), pp 718-725)
Quinoline-8-carboxamides such as 2- and 3-substituted quinoline-8-carboxamides including 2-methylquinoline-8-carboxamide (in particular as disclosed in Lord et al. J. Med. Chem., 2009, 52 (3), pp 868-877)
2-(1-propylpiperidin-4-yl)-1H-benzimidazole-4-carboxamide (A-620223) (Abbott).
aminoethyl pyrrolo dihydroisoquinolinones (in particular as disclosed in Brana et al. Bioorg. Medicin. Chem. Lett. 2009 Vol. 19(15), 4042-4045)
imidazoquinolinone, imidazopyridine and isoquinolindione derivatives (as disclosed in Eltze et al. Mol Pharmacol 2008 vol. 74, 6 1587-1598, in particular 2-[4-(5-Methyl-1H-imidazol-4-yl)-piperidin-1-yl]-4,5-dihydro-imidazo[4,5,1-i,j]quinolin-6-one (BYK49187), 2-(4-pyridin-2-yl-phenyl)-4,5-dihydro-imidazo[4,5,1-i,j]quinolin-6-one (BYK236864), 6-chloro-8-hydroxy-2,3-dimethyl-imidazo-[1,2-α]-pyridine (BYK20370), and 4-(1-methyl-1H-pyrrol-2-ylmethylene)-4H-isoquinolin-1,3-dione (BYK204165))
E7016 (formerly known as GPI21016) (Eisai)
2-[Methoxycarbonyl(4-methoxyphenyl)methylsulfanyl]-1H-benzimidazole-4-carboxylic Acid Amide (KR-33889)
4-Carboxamidobenzimidazole-2-ylpyrroline and -tetrahydropyridine Nitroxides derivatives
N-[3-(4-oxo-3,4-dihydro-phthalazin-1-yl)phenyl]-4-(morpholin-4-yl) butanamide methanesulfonate monohydratate (ONO)
Phenanthridinone
4-iodo-3-nitrobenzamide
2-(4-hydroxyphenyl)-1H-benzimidazole-4-carboxamide
2-aryl-1H-benzimidazole-4-carboxamides such as 2-phenyl benzimidazole 4-carboxamides (WO/09524379)
Phthalazin-1(2H)-one
3-substituted 4-benzyl-2H-phthalazin-1-ones and derivatives (in particular as disclosed in Menear et al. J. Med. Chem., 2008, 51 (20), pp 6581-6591 and Cockcroft et al. Bioorg. Medicin. Chem. Lett. 2006 Vol. 16(4), 1040-1044)
combinations of the above, analogues and derivatives and pharmaceutically acceptable salts thereof, optionally in combination with temozolomide.

Many of the above PARP-inhibitors are for instance commercially available under the brandname Calbiochem from EMD Chemicals Inc., an affiliate of Merck KGaA, Darmstadt, Germany.

The PARP-inhibitors are used in aspects of the invention in a therapeutically effective amount, i.e. in an amount that induces DSBs in the genome of said cells and which is cytotoxic when said cells are subjected to said inhibitors in combination with hyperthermia or in combination with hyperthermia and HSP inhibitors, but that is not necessarily cytotoxic to other cells, and that is not necessarily cytotoxic when HR is not inhibited. These amounts differ between the various PARP-inhibitors as some inhibitors are more potent than others, as evidenced by their respective IC50 values. In addition, the therapeutically effective amount of the PARP-inhibitor will vary from subject to subject depending on the age of the subject, their general health, the severity of the disorder being treated and the mode of administration. It is therefore not possible to specify an exact therapeutically effective amount, however one skilled in the art would be capable of determining a therapeutically effective amount by routine trial and experimentation. Suitable ranges are described herein below.

In aspects of the present invention, the step of inducing inhibition of HR in said cells by hyperthermia will involve elevating the temperature of the cells or tumor to a temperature of between about 41.0 and 43.0° C. It is an aspect of the present invention that this elevated temperature is maintained for at least a duration of time which results in inhibition, inactivation or degradation of BRCA2 or inhibition of BRCA2-mediated DSB repair in said cells. Generally, hyperthermia will be maintained for a period of between 5 to 200 min depending on the exact temperature applied and the type of cell or tissue treated. Very suitably, a temperature of 41° C. will be maintained for about 75 min. The hyperthermia may suitably be performed at a temperature of between 41-42.5° C., most preferably 41-41.5° C.

### BRIEF DESCRIPTION OF THE DRAWINGS

FIG. 1 shows that mild (<43° C.) hyperthermia radiosensitizes HR-proficient, but not HR-deficient cells and inhibits HR repair. a, Cloning efficiency of wild-type (squares), Rad54^{-/-} (circles) and DNA-PKcs^{-/-} (triangles) mouse ES cells incubated for 75 min at 37° C. (open symbols) or 41° C. (filled symbols) and subsequently irradiated with the indicated dose of γ-rays. b, Cloning efficiency of HeLa cells transfected with siRNA directed against luciferase (squares) or XRCC3 (circles), incubated for 75 min at 37° C. (open symbols) or 42.5° C. (filled symbols) and irradiated with the indicated dose of γ-rays. Inset shows reduction of XRCC3 protein levels in HeLa cells transfected with siRNA directed against XRCC3. Cell lysates were analyzed by immunoblotting with antibodies against XRCC3. Equal sample loading was verified by probing for ORC2. c, Efficiency of HR-mediated gene targeting in mouse ES cells. Cells were incubated for 7 h at 37° C. or 41° C. in the presence or absence of 100 nM 17-DMAG. At 2 h into this incubation period cells were transfected with the hRad54GFP-puro knock-in targeting construct. Cells containing integrated construct were then selected in medium containing puromycin and analyzed by FACS. FACS profiles obtained in a single representative experiment (left panel) show the percentage of GFP-positive cells which incorporated the targeting construct into the mRad54 locus after incubation at indicated conditions. The bar graph (right panel) shows relative average percentage of GFP-positive cells obtained from 3 independent experiments. Error bars represent standard error of the mean.

FIG. 2 shows that mild hyperthermia interferes with functions and stability of HR proteins. a, Visualization of accumulation of repair proteins at DSB sites in cells preincubated at 37° C. or 41° C. U2OS cells were incubated at 37° C. or 41° C. for 60 min, then irradiated with a-particles from a source positioned alongside the cells, resulting in linear arrays of DSBs, incubated for 15 min at 37° C. and fixed. Cells were stained for DNA (blue), γH2AX or MDC1 (red), which were used as markers of the DSBs induced by a-particles, and either of the following proteins: MRE11, RPA34, BRCA2 or RAD51 (green). Scale bar—5 μm. b, Quantification of accumulation of repair proteins at DSB sites in cells preincubated at 37° C. or 41° C. Cells treated and prepared as in (a) were scored as positive if they contained at least 3 IRIF of indicated repair protein co-localizing with either γH2AX or MDC1 IRIF. Graphs represent average percentages of positive cells. Error bars represent the range of percentages obtained from 2 independent experiments. At least 50 cells containing damage induced by α-particles were scored per experiment. c, Immunoblotting of cells subjected to mild hyperthermia and/or proteasome inhibitor. HeLa cells were incubated at 37° C. or 42.5° C. for 75 min, in the presence or absence of 10 μM MG132. Next, cells were lysed and lysates were analyzed by immunoblotting with antibodies against RAD51 (upper panel) or BRCA2 (lower panel). Equal sample loading was verified by probing for GRB2 or ORC2. d, Visualization of accumulation of RAD51 at DSB sites in cells isolated from cervix carcinoma biopsies preincubated at 37° C. or 41° C. for 60 min, then irradiated with α-particles from a source positioned above the cells, incubated for 30 min at 37° C. or 41° C. and fixed. Cells were stained for DNA (blue), γH2AX (red) and RAD51 (green). Scale bar—5 μm.

FIG. 3 shows that mild hyperthermia sensitizes cells lacking PARP-1 functionality. a, b, Cloning efficiency of U2OS (a) or R1 (b) cells incubated at 41° C. for the indicated period of time in the absence (open symbols) or presence (filled symbols) of 100 μM NU1025. Graphs represent average cloning efficiencies, corrected for the toxicity of NU1025. Error bars represent standard error of the mean from 3 independent experiments. c, Cloning efficiency of cells with reduced levels of PARP-1 protein subjected to mild hyperthermia. HeLa cells were transfected with siRNA directed against luciferase (circles) or PARP-1 (siRNA #1—triangles, siRNA #2—squares) and incubated at 42.5° C. for the indicated period of time. Error bars represent standard error of the mean in a single experiment. Figure is representative for 3 independent experiments. Inset shows the efficiency of siRNA-induced reduction of PARP-1 protein levels. Cell lysates were analyzed by immunoblotting with antibodies against PARP-1. Equal sample loading was verified by probing for ORC2.

FIG. 4 shows that inhibition of HSP90 enhances hyperthermia-induced degradation of BRCA2 and sensitivity of hyperthermia-treated cells to PARP-1 inhibitors and to ionizing radiation. a, Immunoblotting of cells subjected to mild hyperthermia and/or HSP90 inhibitor. BLM cells have been incubated at 37° C. or 42.5° C. for 60 min in the presence or absence of 100 nM 17-DMAG. Next, cells were lysed and lysates were analyzed by immunoblotting with antibodies against BRCA2. Equal sample loading was verified by probing for ORC2. b, Cloning efficiency of R1 cells incubated for the indicated period of time at 41° C. in the presence or absence of 100 μM NU1025 and/or 100 nM 17-DMAG. Error bars represent the range of cloning efficiencies obtained in 2 independent experiments. c, Cloning efficiency of HeLa cells incubated for 75 min at 42.5 or 37° C. and subsequently irradiated with the indicated dose of γ-rays, in the presence or absence of 100 nM 17-DMAG. Error bars represent standard error of the mean from 3 independent experiments.

FIG. 5 shows that mild hyperthermia interferes with HR. a, Influence of mild hyperthermia on the frequencies of spontaneous and mitomycin C-induced sister-chromatid exchanges. RKO (left panel) or SW-1573 (right panel) cells were incubated for 2 cell cycles in the presence of BrdU, then for 60 min in the presence or absence of mitomycin C at 37° C. or 41° C. and processed to obtain metaphase spreads. Graph presents average number of sister-chromatid exchanges (SCE) per scored metaphase. Error bars indicate standard error of the mean obtained from 3 independent experiments. b, Influence of mild hyperthermia on accumulation of RAD54-GFP at DSB sites. Mouse knock-in ES cells expressing RAD54-GFP were incubated at 37° C. or 41° C. for 75 min, then irradiated with γ-rays (8Gy, left column) or α-particles (right column) and fixed 30 min after irradiation. Cells were then either directly imaged using a confocal microscope, (left column) or stained for γH2AX and DNA and imaged using a wide-field fluorescence microscope (right column). Scale bar—5 μm. c, Quantification of accumulation of GFP-RAD51 and EGFP-KU80 at sites of DNA damage induced by UVA laser microirradiation in living cells preincubated at 37° C. or 41° C. V79 cells expressing GFP-RAD51 (left panel) and XR-V15B cells expressing EGFP-KU80 (right panel) were incubated at 37° C. or 41° C. for 60 min, then exposed to UVA light in predefined areas of the nuclei and imaged for indicated period of time. Graphs represent mean increase of fluorescence in the exposed areas as a function of time. Error bars represent standard deviation around the mean from at least 10 measurements.

FIG. 6 shows that mild hyperthermia induces degradation of BRCA2. a, Immunoblotting of cells subjected to mild hyperthermia. BLM cells have been incubated at 37° C. or 42.5° C. for 60 min. Next, cells were incubated for the indicated period of time at 37° C. and lysed. Lysates were analyzed by immunoblotting with antibodies against BRCA2 (upper panel). Equal sample loading was verified by probing for ORC2 (lower panel). b, Immunoblotting of skin cells subjected to mild hyperthermia. Freshly isolated human skin fragments were incubated at 42.5° C. or 37° C. for 75 min and lysed. Lysates were analyzed by immunoblotting with antibodies against BRCA2 (upper panel). Equal sample loading was verified by Coomassie staining after transfer (lower panel).

### DETAILED DESCRIPTION OF THE INVENTION

### Definitions

In the context of this specification, the term “hyperthermia” as used herein, refers to a type of treatment in which body tissue is exposed to high temperatures (generally 45-46° C.) to damage and kill (cancer) cells or to make cancer cells more sensitive to the effects of ionizing radiation and certain anticancer drugs. With “mild hyperthermia” is meant a treatment comprising exposing the tissue to a heat source that results in a temperature of the tissue of between about 41.0-45.0° C. In general, the hyperthermia involves only a local treatment, wherein heat is applied to a small area or region, such as a tumor, using probes for introduction into tissue that deliver energy only to a specific target tissue, while essentially leaving the surrounding non-target tissue unaffected. However, in aspects of the present invention regional (large areas of tissue, such as a body cavities, organs, or limbs) or whole body hyperthermia can also be applied. The temperature of the treatment area can be monitored using thermometers inserted into the tissue to be treated. Imaging techniques, such as computed tomography, may be used to make sure the probes are properly positioned. In order to heat the tissue, radiation (e.g. electromagnetic energy radiation), microwave, radiofrequency, ultrasound and other methods of heating tissue can be used. Hyperthermia can be provided using dedicated instrumentations for instance as described in U.S. Pat. No. 5,412,182, wherein eddy currents are generated within a metallic needle tube, U.S. Pat. No. 6,904,323, wherein antennas transmitting RF energy are used, and WO 02/45790, wherein a microwave antenna is used. Generally, the device is comprised of a needle-like probe serving as a microwave antenna. Microwaves are emitted from the probe to increase the temperature of cancerous body tissue. A typical device includes a probe having a needle-like distal tip sharp enough to pierce and penetrate skin, tissue and cancerous clusters, such as tumors. For the most effective treatment, the probe is implanted approximately across the center of the tumor to be treated. However, the probe may also be placed in the proximity of the tumors, such that many cancerous conglomerates can be simultaneously treated. The probe that serves as a microwave emitter is connected to a microwave generator. The microwave generator may continuously or intermittently generate microwaves at an adjustable frequency, such as 1.5 GHz. The frequency of the microwaves is usually in the range of 1-5 GHz. An optimal operating frequency can be selected to achieve penetration of the microwave radiation into the tissue surrounding the probe, adequate for heating a particular sized tumor. Microwave generators are commercially available, for instance from Matsushita Electric Industries, Ltd. of Tokyo, Japan. A generator can be modified so as to produce lower power output levels sufficient for the purposes to which the present invention is to be placed. Typically a microwave generator generates 15-100 watts of power, which is emitted through the probe into the surrounding tissue, warming the tissue.

Local hyperthermia can also be provided using external approaches, intraluminal or endocavitary methods, or interstitial techniques. External approaches are used to treat tumors that are in or just below the skin. External applicators are positioned around or near the appropriate region, and energy is focused on the tumor to raise its temperature. Intraluminal or endocavitary methods may be used to treat tumors within or near body cavities, such as the esophagus or rectum. Probes are placed inside the body cavity and inserted into the tumor to deliver energy and heat the area directly. Interstitial techniques as described in detail above are used to treat tumors deep within the body, such as brain tumors. This technique allows the tumor to be heated to higher temperatures than external techniques. Under anesthesia, probes or needles are inserted into the tumor. Imaging techniques, such as ultrasound, may be used to make sure the probe is properly positioned within the tumor.

In the context of this specification, a “condition associated with hyperproliferative cellular division” refers to any clinical condition characterised by or otherwise involving an increased rate of cell division relative to a normal (healthy) reference rate. Conditions associated with hyperproliferative cellular division include, but are not limited to: myeloproliferative syndromes such as Langerhans cell histiocytosis, mastocytosis, mixed myeloproliferative and myelodysplastic conditions, dermal proliferative conditions such as psoriasis, non-bullous congenital ichthyosiform erythroderma. Conditions associated with hyperproliferative cellular division also include cancer, whether benign or malignant, including haematopoietic malignant cancers.

The terms “hyperproliferation” and “hyperproliferating” refer to the abnormal growth of a cell type, which can be cancerous or benign. Generally, hyperproliferating cells exhibit a rate of cell division that is at least about ten percent greater than the rate of cell division exhibited by normal cells of that cell type.

In the context of this specification, the terms “treatment” and “treating” refer to any and all uses which remedy a condition or disease or symptoms thereof, prevent the establishment of a condition or disease or symptoms thereof, or otherwise prevent or hinder or reverse the progression of a condition or disease or other undesirable symptoms in any way whatsoever.

In the context of this specification, the term “therapeutically effective amount” includes within its meaning an amount of the active agent or treatment sufficient to provide the desired therapeutic effect. The exact amount will vary from subject to subject depending on the age of the subject, their general health, the severity of the disorder being treated and the mode of administration. It is therefore not possible to specify an exact “therapeutically effective amount”, however one skilled in the art would be capable of determining a “therapeutically effective amount” by routine trial and experimentation. A therapeutically effective amount is preferably essentially non-lethal to normal, non-tumor cells. Under the conditions wherein BRCA2 is inhibited, the therapeutically effective amount has a cytotoxic effect and represents a cytotoxic amount with respect to the target cells.

In the context of this specification, the term “cytotoxic amount” is defined to mean an amount of a cytotoxic agent that is toxic, i.e. essentially lethal, to the target cell once the cytotoxic agent or combination of agents have reached the cell. Generally, toxicity is indicated by statistically significant loss in cell viability. In the context of the present invention a ‘cytotoxic amount’ of a DSB-inducing agent such as a PARP-1 inhibitor may include reference to a non-toxic amount with respect to normal cells, whereas the amount will nevertheless kill cells that are rendered more sensitive to these inhibitors by hyperthermia. Hence, a cytotoxic amount of an agent that induces double strand breaks in the DNA of hyperproliferative cells refers to a therapeutically effective amount in the context of the combination anti-cancer therapy contemplated herein. A cytotoxic amount of a DSB inducing agent is therefore an amount of said agent that is cytotoxic to cells that have been rendered sensitive to said agent by hyperthermia as described herein.

The term “chemotherapeutic agent” refers to a chemical compound useful in the treatment of cancer or other condition characterized by a hyperproliferation of cells.

In the context of this specification, “pharmaceutically acceptable salts” include, but are not limited to, those formed from: acetic, ascorbic, aspartic, benzoic, benzenesulfonic, citric, cinnamic, ethanesulfonic, fumaric, glutamic, glutaric, gluconic, hydrochloric, hydrobromic, lactic, maleic, malic, methanesulfonic, naphthoic, hydroxynaphthoic, naphthalenesulfonic, naphthalenedisulfonic, naphthaleneacrylic, oleic, oxalic, oxaloacetic, phosphoric, pyruvic, p-toluenesulfonic, tartaric, trifluoroacetic, triphenylacetic, tricarballylic, salicylic, sulfuric, sufamic, sulfanilic and succinic acid.

### Preferred Embodiments

The present inventors have discovered that mild (41-42.5° C.) hyperthermia inhibits the HR pathway of DSB repair by inducing degradation and/or inactivation of BRCA2 and that this effect can be significantly enhanced by inhibition of Hsp90. Mammalian tumour cells deficient in HR are sensitive to the inhibition of PARP1. It has now been demonstrated that hyperthermia renders tumour cells with an intact HR pathway sensitive to PARP inhibition, even when said cells are not BRCA1 or BRCA2 deficient, enabling design of novel therapeutic strategies involving localized induction of HR deficiency.

In one aspect, the present invention relates to a method for inhibiting HR in cells comprising inducing the degradation, inactivation and/or inhibition of BRCA2 in said cells by hyperthermia.

A method for inhibiting HR according to the present invention comprises subjecting the cells to hyperthermia to the extent that BRCA2 is essentially completely degraded, inhibited or inactivated, or at least to the extent that the levels of BRCA2 in the cell are substantially lowered. In therapeutic applications, the exact temperature of the hyperthermia is selected such that an optimum is sought between the level of inhibition of HR via degradation, inhibition or inactivation of BRCA2 in the target cells and the damage to non-target cells due to the heat treatment. In fact, it is preferred that the temperature in therapeutic applications of inhibiting HR is selected to correspond to the lowest temperature which still results in a therapeutically effective inhibition of HR in the target cells in a treatment period of about 1 minute to 4 hours, preferably in a period of between 15 and 180 min. The inhibition of HR being therapeutically effective when the target cells are rendered sensitive to PARP1, which according to the present invention is achieved when BRCA2 is degraded, inhibited or inactivated to a level at which BRCA2-mediated repair of DSBs is inhibited or prevented such that PARP1 kills the cells. This allows for an optimization of the temperature and the duration of hyperthermia that minimizes injury to non-target tissues while maximizing impairment of HR-mediated DNA repair.

The level of degradation, inhibition or inactivation of BRCA2 can be determined by measuring BRCA2 protein levels in the cells of interest (i.e. the BRCA2 protein content in cells), for instance by immunoblotting using anti-BRCA2 antibodies or by analysis of accumulation of BRCA2 at DSB sites by immunochemistry, as described in the Examples below. Immunoblotting usually includes SDS-PAGE gel electrophoresis to separate cell proteins and transfer of the proteins to a membrane. Immunochemistry usually involves staining of irradiated cells with antibodies against protein of interest and microscopical analysis.

A method of the invention for inhibiting HR in cells comprising inducing the degradation or inhibition of BRCA2 in said cells by hyperthermia can in principle be applied to any cell or tissue. The method will usually involve the steps of determining the optimal duration and temperature of hyperthermia in the target cells or tissue in which HR is to be inhibited as described above. This can be achieved for various tumor types by empirical testing in order to determine the optimal duration of the treatment. The optimal treatment duration is determined by the steps of subjecting the target cells or tissue in which HR is to be inhibited to hyperthermia at a fixed temperature, for instance 41° C., and determining the level of BRCA2 in those cells or tissues at regular time intervals in order to determine the period of time required from the onset of the treatment to achieve degradation, inhibition or inactivation of BRCA2 in the said target cells or tissue or to achieve at least a significant reduction in the level of BRCA2 protein in said cells. The method of the invention then comprises the step of subjecting the target cells or tissue in which HR is to be inhibited to hyperthermia at 41° C. for at least the period of time thus determined, and preferably not longer.

Degradation, inhibition, or inactivation of BRCA2, or reduction in the level of BRCA2 is to be achieved at a level at which DSB repair is inhibited or prevented. This level is suitable determined prior to or parallel to the actual treatment in a test-treatment, for the specific cell or tumor type, wherein the hyperthermia is dosed at varying intensities under simultaneous application of DSB inducing agents or conditions wherein the effect of hyperthermia on cell or tumor viability is tested in a dose-dependent manner, and wherein the killing of the cells or tumor indicates that the cells or tumor are rendered sensitive to the DSB inducing agent (eg. PARP1). The term “DSB inducing agent” as used herein includes reference to DSB inducing therapies, such as ionizing radiation, or other DSB inducing treatments, but preferably relates to chemical treatments, and most preferably PARP inhibitors.

In another aspect, the present invention provides a method of treating or preventing hyperproliferative disease in a body tissue of a subject, comprising the steps of:

a) administering (locally or systemically) to a subject in need thereof a therapeutically effective amount of an agent that induces DSBs in the DNA of the hyperproliferative cells of said body tissue; and

b) subjecting the hyperproliferative cells of said body tissue to hyperthermia prior to, simultaneously with or subsequent to step a) to thereby degrade, inhibit, inactivate or reduce levels of BRCA2 in said cells.

In such a method, the hyperthermia is used to block HR in the hyperproliferative cells of said body tissue. Hence, it is preferred that the degradation, inhibition or inactivation of BRCA2 in said cells is essentially complete, or that at least the level of BRCA2 protein in the cells is reduced to such extent that HR is prevented, or cells are rendered sensitive to PARP1 such that when said cells are exposed to PARP1, said PARP1 is cytotoxic.

In aspects of the present invention, the cells may be any mammalian tissue cell. Preferably, the cell is not deficient in the Breast Cancer Susceptibility Protein BRCA2. In fact, in certain preferred embodiments of aspect of the present invention, breast cancer cells genetically defective in BRCA2 are disclaimed. However, in other preferred embodiments, hyperproliferative cells comprise cells of any cancer, preferably cells of a solid tumor. The cancer may be selected from the group which includes, but which is not limited to, gastrointestinal tumours, cancer of the liver and biliary tract, pancreatic cancer, prostate cancer, testicular cancer, blood cancer, lung cancer, skin cancer (for example melanoma), breast cancer, non-melanoma skin cancer (for example basal cell carcinoma and squamous cell carcinoma), ovarian cancer, uterine cancer, cervical cancer, cancer of the head and neck, bladder cancer, sarcomas and osteosarcomas, Kaposi sarcoma, AIDS-related Kaposi sarcoma and renal carcinoma.

In aspects of the present invention, the agent that induces DSBs in the

DNA of the hyperproliferative cells is preferably a chemical agent, alternatively it may include ionizing radiation. Most preferably the agent is a PARP inhibitor. Suitable PARP inhibitors include both PARP-1 and PARP-2 inhibitors. Examples thereof include, but are not limited to 5-aminoisoquinolinone; 3-methyl-5-aminoisoquinolinone; 3-aminobenzamide; 5-iodo-6-amino-1,2-benzopyrone; 3,4-dihydro-5[4-(1-piperindinyl)butoxy]-1(2H)-isoquinoline; 1,5-dihydroxyisoquinoline; -aza-5[H]-phenanthridin-6-ones; 6(5H)-phenanthridinone; 4-amino-1,8-naphthalimide; 8-hydroxy-2-methylquinazoline-4-one; N-(6-oxo-5,6-dihydrophenanthridin-2-yl)-N,N-dimethylacetamide; indeno-isoquinolinone; 5-chloro-2-[3-(4-phenyl-3,6-dihydro-1(2H)-pyridinyl)propyl]-4(3H)-quinazolinone; 1-piperazineacetamide, 4-[1-(6-amino-9H-purin-9-yl)-1-deoxy-6-D-ribofuranuron]-N-(2,3-dihydro-1H-isoindol-4-yl)-1-one; thieno[2,3-c]isoquinolin-5-one; 2-dimethylaminomethyl-4H-thieno [2,3-c]isoquinolin-5-one; 4-hydroxyquinazoline; nicotinamide; minocycline; 2-methyl-3,5,7,8-tetrahydrothiopyrano[4,3-d]pyrimidine-4-one; 3-(4-chlorophenyl)quinoxaline-5-carboxamide; benzamide; N-(6-oxo-5,6-dihydrophenanthridin-2-yl)-2-(N,N-dimethylamino)acetamide; AG014699; AG14361; 2-[(R)-2-methylpyrrolidin-2-yl]-1H-benzimidazole-4-carboxamide; 4-[3-(4-cyclopropanecarbonylpiperazine-1-carbonyl)-4-fluorobenzyl]-2H-phthalazin-1-one; BSI-401; BSI-201; CEP-8983; CEP-9722; GPI-21016; GPI 16346; GPI 18180; GPI 6150; GPI 18078; GPI 6000; 2-aminothiazole analogues; quinoline-8-carboxamides; 2- and 3-substituted quinoline-8-carboxamides; 2-methylquinoline-8-carboxamide; 2-(1-propylpiperidin-4-yl)-1H-benzimidazole-4-carboxamide; aminoethyl pyrrolo dihydroisoquinolinones; imidazoquinolinone and derivatives thereof; imidazopyridine and derivatives thereof; isoquinolindione and derivatives thereof; 2-[4-(5-Methyl-1H-imidazol-4-yl)-piperidin-l-yl]-4,5-dihydro-imidazo[4,5,1-i,j]quinolin-6-one; 2-(4-pyridin-2-yl-phenyl)-4,5-dihydro-imidazo[4,5,1-i,j]quinolin-6-one; 6-chloro-8-hydroxy-2,3-dimethyl-imidazo-[1,2-α]-pyridine; 4-(1-methyl-1H-pyrrol-2-ylmethylene)-4H-isoquinolin-1,3-dione; E7016; 2-[methoxycarbonyl(4-methoxyphenyl)methylsulfanyl]-1H-benzimidazole-4-carboxylic Acid Amide; 4-carboxamidobenzimidazole-2-ylpyrroline; tetrahydropyridine nitroxides derivatives; N-[3-(4-oxo-3,4-dihydro-phthalazin-1-yl)phenyl]-4-(morpholin-4-yl) butanamide methanesulfonate monohydratate; phenanthridinone; 4-iodo-3-nitrobenzamide; 2-(4-hydroxyphenyl)-1H-benzimidazole-4-carboxamide; 2-aryl-1H-benzimidazole-4-carboxamides; 2-phenyl benzimidazole 4-carboxamides; phthalazin-1(2H)-one; 3-substituted 4-benzyl-2H-phthalazin-1-ones and derivatives; combinations of the above, analogues and derivatives and pharmaceutically acceptable salts thereof. As the DSB-inducing agent also combinations of PARP-1 and/or PARP-2 inhibitors may be used, as well as any pharmaceutically acceptable salts thereof.

The PARP inhibitor is suitably applied locally, but may also be administered systemically (i.e. throughout the body). In any form, the PARP inhibitor is suitably provided in the form of a pharmaceutical composition, comprising the PARP inhibitor and a pharmaceutically acceptable carrier.

The optional HSP inhibitor is also suitably applied locally, but may also be administered systemically (i.e. throughout the body). In any form, the HSP inhibitor is suitably provided in the form of a pharmaceutical composition, comprising the HSP inhibitor and a pharmaceutically acceptable carrier.

In composition described below, the PARP inhibitor and/or the HSP inhibitor is/are meant when using the term “active agent(s)”.

Pharmaceutical compositions include those suitable for oral, parenteral (including subcutaneous, intradermal, intramuscular, intravenous and intraarticular), inhalation (including use of metered dose pressurised aerosols, nebulisers or insufflators), rectal and topical (including dermal, buccal, sublingual and intraocular) administration.

The compositions may conveniently be presented in unit dosage form and may be prepared by any of the methods well known in the art of pharmacy. All methods include the step of bringing one or more of the active agents into association with a carrier which constitutes one or more accessory ingredients. In general, the compositions are prepared by uniformly and intimately bringing into association one or more of the active agents with a liquid carrier or finely divided solid carrier, or both and then, if necessary, shaping the product into the desired composition.

Generally, a therapeutically effective dosage of the active agents is expected to be in the range of about 0.0001 mg to about 1000 mg per kg body weight per 24 hours; about 0.001 mg to about 750 mg per kg body weight per 24 hours; about 0.01 mg to about 500 mg per kg body weight per 24 hours; about 0.1 mg to about 500 mg per kg body weight per 24 hours; about 0.1 mg to about 250 mg per kg body weight per 24 hours, or about 1.0 mg to about 250 mg per kg body weight per 24 hours. More typically, an effective dose range is expected to be in the range of about 1.0 mg to about 200 mg per kg body weight per 24 hours; about 1.0 mg to about 100 mg per kg body weight per 24 hours; about 1.0 mg to about 50 mg per kg body weight per 24 hours; about 1.0 mg to about 25 mg per kg body weight per 24 hours; about 5.0 mg to about 50 mg per kg body weight per 24 hours; about 5.0 mg to about 20 mg per kg body weight per 24 hours, or about 5.0 mg to about 15 mg per kg body weight per 24 hours.

Alternatively, a therapeutically effective dosage of the active agents may be up to about 500 mg/m². Generally, an effective dosage of the active agents is expected to be in the range of about 25 to about 500 mg/m², about 25 to about 350 mg/m², about 25 to about 300 mg/m², about 25 to about 250 mg/m², about 50 to about 250 mg/m², or about 75 to about 150 mg/m².

Compositions suitable for buccal (sublingual) administration include lozenges comprising the active agents in a flavoured base, usually sucrose and acacia or tragacanth; and pastilles comprising the active agents in an inert base such as gelatine and glycerin or sucrose and acacia.

Compositions suitable for oral administration may be presented as discrete units such as gelatine or HPMC capsules, cachets or tablets, each containing a predetermined amount of the active agents as a powder, granules, as a solution or a suspension in an aqueous liquid or a non-aqueous liquid, or as an oil-in-water liquid emulsion or a water-in-oil liquid emulsion. The active agents may also be present as a paste.

When the active agents are formulated as capsules, the compound may be formulated with one or more pharmaceutically acceptable carriers such as starch, lactose, microcrystalline cellulose, silicon dioxide and/or a cyclic oligosaccaride such as cyclodextrin. Suitable cyclodextrins include α-cyclodextrin, β-cyclodextrin, γ-cyclodextrin, 2-hydroxyethyl-β-cyclodextrin, 2-hydroxypropyl-cyclodextrin, 3-hydroxypropyl-β-cyclodextrin and tri-methyl-γ-cyclodextrin. The cyclodextrin may be hydroxypropyl-β-cyclodextrin. Suitable derivatives of cyclodextrins include Captisol® a sulfobutyl ether derivative of cyclodextrin and analogues thereof as described in U.S. Pat. No. 5,134,127.

Tablets may be prepared by compression or moulding, optionally with one or more accessory ingredients. Compressed tablets may be prepared by compressing in a suitable machine the active agents in a free-flowing form such as a powder or granules, optionally mixed with a binder, lubricant (for example magnesium stearate or calcium stearate), inert diluent or a surface active/dispersing agent. Moulded tablets may be made by moulding a mixture of the powdered active agents moistened with an inert liquid diluent, in a suitable machine. The tablets may optionally be coated, for example, with an enteric coating and may be formulated so as to provide slow or controlled release of the active agents therein.

Compositions for parenteral administration include aqueous and non-aqueous sterile injectable solutions which may contain anti-oxidants, buffers, bacteriostats and solutes which render the formulation isotonic with the blood of the intended recipient, and which may include suspending agents and thickening agents. A parenteral composition may comprise a cyclic oligosaccaride such as hydroxypropyl-β-cyclodextrin. The compositions may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use.

Compositions suitable for transdermal administration may be presented as discrete patches adapted to remain in intimate contact with the epidermis of the recipient for a prolonged period of time. Such patches suitably comprise the active agents as an optionally buffered aqueous solution of, for example, 0.1 M to 0.2 M concentration with respect to the active agents.

Compositions suitable for transdermal administration may also be delivered by iontophoresis, and typically take the form of an optionally buffered aqueous solution of the active agents. Suitable compositions may comprise citrate or Bis/Tris buffer (pH 6) or ethanol/water and contain from 0.1 M to 0.2 M of the active agents.

Spray compositions for topical delivery to the lung by inhalation may, for example be formulated as aqueous solutions or suspensions or as aerosols, suspensions or solutions delivered from pressurised packs, such as a metered dose inhaler, with the use of a suitable liquefied propellant. Suitable propellants include a fluorocarbon or a hydrogen-containing chlorofluorocarbon or mixtures thereof, particularly hydrofluoroalkanes, e.g. dichlorodifluoromethane, trichlorofluoromethane, dichlorotetrafluoroethane, especially 1,1,1,2-tetrafluoroethane, 1,1,2,2,3,3,3-heptafluoro-n-propane or a mixture thereof. Carbon dioxide or other suitable gas may also be used as propellant. The aerosol composition may be excipient free or may optionally contain additional composition excipients well known in the art, such as surfactants e.g. oleic acid or lecithin and cosolvents e.g. ethanol. Pressurised compositions will generally be retained in a canister (e.g. an aluminium canister) closed with a valve (e.g. a metering valve) and fitted into an actuator provided with a mouthpiece.

Medicaments for administration by inhalation desirably have a controlled particle size. The optimum particle size for inhalation into the bronchial system is usually 1-10 μm, preferably 2-5 μm. Particles having a size above 20 μm are generally too large when inhaled to reach the small airways. When the excipient is lactose it will typically be present as milled lactose, wherein not more than 85% of lactose particles will have a MMD of 60-90 μm and not less than 15% will have a MMD of less than 15 μm.

Compositions for rectal administration may be presented as a suppository with carriers such as cocoa butter or polyethylene glycol, or as an enema wherein the carrier is an isotonic liquid such as saline. Additional components of the compositions may include a cyclic oligosaccaride, for example, a cyclodextrin, as described above, such as hydroxypropyl-β-cyclodextrin, one or more surfactants, buffer salts or acid or alkali to adjust the pH, isotonicity adjusting agents and/or anti-oxidants.

Compositions suitable for topical administration to the skin preferably take the form of an ointment, cream, lotion, paste, gel, spray, aerosol, or oil. Carriers which may be used include Vasoline, lanoline, polyethylene glycols, alcohols, and combination of two or more thereof. The active agents are generally present at a concentration of from 0.1% to 20% w/w, or from 0.5% to 5% w/w each. Examples of such compositions include skin creams.

The composition may also be administered or delivered to target cells in the form of liposomes. Liposomes are generally derived from phospholipids or other lipid substances and are formed by mono- or multi-lamellar hydrated liquid crystals that are dispersed in an aqueous medium. Specific examples of liposomes that may be used to administer or deliver an active agent include synthetic cholesterol, 1,2-distearoyl-sn-glycero-3-phosphocholine, 3-N-[(-methoxy poly(ethylene glycol)2000)carbamoyl]-1,2-dimyrestyloxy-propylamine (PEG-cDMA) and 1,2-di-o-octadecenyl-3-(N,N-dimethyl)aminopropane (DODMA).

The compositions may also be administered in the form of microparticles. Biodegradable microparticles formed from polylactide (PLA), polylactide-co-glycolide (PLGA), and \acute{ε}-caprolactone have been extensively used as drug carriers to increase plasma half life and thereby prolong efficacy (R. Kumar, M., 2000, J Pharm Pharmaceut Sci. 3(2) 234-258).

The compositions may incorporate a controlled release matrix that is composed of sucrose acetate isobutyrate (SAIB) and organic solvent or organic solvent mixtures. Polymer additives may be added to the vehicle as a release modifier to further increase the viscosity and slow down the release rate. The active agents may be added to the SAIB delivery vehicle to form SAIB solution or suspension compositions. When the formulation is injected subcutaneously, the solvent diffuses from the matrix allowing the SAIB-drug or SAIB-drug-polymer mixtures to set up as an in situ forming depot.

In aspects of the present invention the step of subjection the cells or tissue to hyperthermia may constitute raising the temperature in the cells or tissue to any temperature above 40° C. and 46° C. In preferred embodiment, mild hyperthermia (<43° C.) is used. Suitable temperatures include 41.0, 41.1, 41.2, 41.3, 41.4, 41.5, 41.6, 41.7, 41.8, 41.9, 42.0, 42.1, 42.2, 42.3, 42.4, 42.5, 42.6, 42.7, 42.8, and 42.9° C. The temperature can be constant for the duration of the treatment or can be applied dynamically, for instance pulsed. Most preferred ranges include between 41 and 42° C.

In aspects of the invention the method may further comprise administering to a subject in need thereof a therapeutically effective amount of a HSP inhibitor. The HSP inhibitor was found to further sensitize the cells to the DSB-inducing agent. The HSP inhibitor may be any HSP inhibitor, including, but not limited to N-formyl-3,4-methylenedioxy-benzylidene-butyrolactam, 3,4-Methylenedioxy-benzylidine-g-butyrolactam, geldanamycin and derivatives thereof, 17-(Allylamino)-17-demethoxygeldanamycin (17-AAG), 17-dimethylaminoethylamino-17-demethoxy-geldanamycin (17-DMAG), 17-allylamino-17-demethoxygeldanamycin hydroquinone hydrochloride, radicicol, pochonin, radester, 8-arylsulfanyl adenine derivatives, 3,4-diaryl pyrazole resorcinol derivatives, shepherdin and derivatives thereof, retaspimycin hydrochloride, (−)-Epigallocatechin-3-gallate, 4,5-diarylisoxazole derivatives. A highly preferred HSP inhibitor is a HSP90 inhibitor. Very good results have been obtained with 17-DMAG. The HSP inhibitor is applied in a therapeutically effective amount. Said amount is from about 0.0001 mg to about 1000 mg per kg body weight of said subject per 24 hours.

In another aspect, the present invention provides a method of killing cells. The method of the present invention has the advantage that not only BRCA2- or BRCA1-deficient cancer cells can now be killed by PARP inhibitors, but in fact any cell can be transformed into a BRCA2-deficient cell by a method involving hyperthermia as described herein. Once BRCA2-deficient, the cells can be easily killed using a suitable PARP-inhibitor. Hence, a broad method for killing cells comprising the step of (a) administering to the cells an amount of a PARP-inhibitor that is cytotoxic to the cells in combination with hyperthermia. This amount is essentially the amount generally applied to kill BRCA2 deficient breast cancer cells, but will largely depend on the PARP-inhibitor used and the cell type to be killed. A suitable amount is about 0.0001 mg to about 1000 mg per kg body weight of said subject per 24 hours. In vitro testing may be used to determine the optimum amount for specific cell types. IC50 values provide a proper starting point. The skilled person can optimize the amount of PARP-inhibitor that is cytotoxic to cells that have been rendered sensitive to PARP-inhibitors by hyperthermia as described herein, through routine experimentation. Hence, a broad method for killing cells further comprises the step of b) subjecting the cells to hyperthermia to thereby induce in said cells the degradation, inhibition and/or inactivation of BRCA2.

In principle, the hyperthermia treatment can be performed prior to, simultaneously with, or subsequent to exposure of the cells to the PARP-inhibitor. It is preferred in aspects of the invention that the hyperthermia is effected and has rendered the cells sensitive to PARP inhibitors before the administration of the PARP inhibitor to the subject, or the exposure of the cells to the PARP-inhibitor. However, the skilled person will be capable of determining the optimal moment of providing hyperthermia, with reference to the moment of application of the pharmaceuticals, so as to provide the pharmaceuticals the proper window of activity.

Methods of the invention preferably comprise a step wherein the level or activity of BRCA2 in the target cells or tumor is determined.

In a final aspect, the present invention provides the use of a PARP-inhibitor, or a pharmaceutically acceptable salt thereof, for the manufacture of a medicament for killing a cell or retarding the growth of a tumor within a subject in accordance with a treatment regimen involving (a) administering to the cell or tumor within the subject an amount of the medicament comprising said PARP-inhibitor that is cytotoxic to cells prior to, simultaneously or subsequently subjected to hyperthermia and (b) prior to, simultaneously with or subsequently to step (a) inducing in said cell or tumor the degradation, inhibition and/or inactivation of BRCA2 by hyperthermia. The preferred embodiments of this aspect are identical to those described for the other aspects above.

In the treatment of cancer, therapeutic advantages may be obtained through combination treatment regimens. As such, methods of treatment according to the present invention may be used in conjunction with other therapies, such as radiotherapy, chemotherapy, surgery, or other forms of medical intervention. Non-limiting examples of suitable chemotherapeutic and other anti-cancer agents include: taxol, fluorouracil, cisplatin, oxaliplatin, α-interferon, vincristine, vinblastine, angioinhibins, doxorubicin, Neomycin, mitomycin C, phenoxodiol, methramycin, TNP-470, pentosan polysulfate, tamoxifen, LM-609, CM-101 and SU-101.

The co-administration of the active agents and chemotherapeutic or other anti-cancer agents may be simultaneous or sequential. Simultaneous administration may be effected by the active agents being in the same unit dose as a chemotherapeutic or other anti-cancer agent, or the active agents and the chemotherapeutic or other anti-cancer agents may be present in individual and discrete unit doses administered at the same, or at a similar time. Sequential administration may be in any order as required.

All publications mentioned in this specification are herein incorporated by reference. The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present invention will now be further described in greater detail by reference to the following specific examples, which should not be construed as in any way limiting the scope of the invention.

### EXAMPLES

### Example 1

### Inhibition of BRCA2-Mediated Double-Strand Break Repair in Mammalian Cells

### Cell Culture.

Embryonic stem (ES) cells were cultured on gelatin-coated dishes in a 1:1 mixture of Dulbecco's modified Eagle's medium (DMEM) and buffalo rat liver conditioned medium, supplemented with 10% FBS (Hyclone), 0.1 mM nonessential amino acids (Biowhittaker), 50 mM β-mercaptoethanol (Sigma) and 500 U ml-1 leukemia inhibitory factor. Other cells were cultured in following media, supplemented with 10% (v/v) FCS and streptomycin/penicillin: 1:1 mixture of DMEM and Ham's F10 (HeLa), DMEM (human melanoma [BLM], osteosarcoma [U2OS], cervix carcinoma cells), L-15 (human squamous lung carcinoma [SW-1573]), Eagle's MEM (mouse osteosarcoma [MOS], rat rhabdomyosarcoma [R1]). Cells were maintained at 37° C. in an atmosphere containing 5% (HeLa, BLM), 10% (U2OS, cervix carcinoma cells), 2% (R1) or 0% (SW-1573) CO₂. Patients with cervical cancer expressed written informed consent to provide fresh biopsies during an investigation under general anesthesia, and use the tumor specimens for this study. This study was approved by the medical ethical committee of the AMC (MEC #03/137). The biopsies were minced using scalpel, then incubated in Liberase Blendzyme enzyme cocktail (Roche) in DMEM for 60 min at 37° C. and plated on glass coverslips in fresh culture medium. All incubations at elevated temperature were performed in a heated waterbath or in a water-filled cuvette placed in an incubator set to required temperature, in an atmosphere containing appropriate CO2 concentration.

### Antibodies.

Following antibodies were used for western Notting: rabbit (#PC146) and mouse (#OP95) anti-BRCA2 (Calbiochem), rabbit anti-hRAD51 (37), rabbit anti-XRCC3 (ab6494, Abcam), rabbit anti-hORC2 (#559266) and mouse anti-hGRB2 (#610112) (BD Pharmingen), mouse anti-hPARP-1 (C2-10, Alexis Biochemicals) and relevant horseradish peroxidase-conjugated secondary antibodies (Jackson Immunoresearch). Following antibodies were used for immunofluorescence: mouse-anti hBRCA2 (Ab-1, Calbiochem), mouse-anti hRPA34 (Ab-2, Oncogene), mouse anti-hγH2AX (05-636, Milipore), rabbit anti-hMDC1 (A300-051A, Bethyl Laboratories), rabbit anti-hRAD51 (37), rabbit anti-hMRE11 (38), goat anti-mouse-Cy3, (115-165-166) and goat anti-rabbit-FITC (111-095-144) (Jackson Immunoresearch).

### Inhibitors.

The following inhibitors were used at the indicated final concentrations: 17-DMAG, 100 nM (Sigma-Aldrich), NU1025, 100 μM (Sigma-Aldrich), MG-132, 10 μM (Calbiochem).

### Tissue/Cell Lysis and Immunoblotting.

Cells were lysed in SDS sample buffer (2% SDS, 10% glycerol, 60 mM Tris-HCl pH 6.8). Skin tissue was lysed in SDS sample buffer, supplemented with protease inhibitor (Roche) and homogenized 2×3 min at 30 Hz in Tissue Lyser II (Qiagen) with 5 mm stainless steel beads (Qiagen), on ice. After centrifugation, the supernatant was supplemented with Benzonuclease (Merck), incubated for 10 min at room temperature and 5 min at 95° C. Protein concentration was determined by the Lowry protein assay, extracts were supplemented with 0.5% 6-mercaptoethanol and 0.02% bromophenol blue. After fractionation by SDS-PAGE, proteins were transferred to a nitrocellulose membrane and probed with the relevant antibodies. For immunoblotting of BRCA2, a 3-8% Tris-acetate gel system was used. BRCA2 was transferred onto a PVDF membrane.

siRNA Treatment.

Transfection of siRNA duplexes was carried out using Lipofectamine 2000 (Invitrogen) according to the manufacturer's instructions. Cells transfected with 200 pmol siRNA per 60-mm culture dish were used for cloning efficiency assays or immunoblotting 48 h after transfection. Sense sequences of siRNA were GAAAGUGUGUUCAACUAAUUU (#1) and GCAACAACUGGAACAGAUU (#2) against Parp-1, GGACCUGAAUCCCAGAAUUUU against XRCC3 and CGUACGCGGAAUACUUCGAdTdT against luciferase.

### Clonogenic Assays.

In experiments involving NU1025 or irradiation, cells were trypsinized, counted and plated at appropriate concentrations into 60- or 35 mm dishes. After a 4-6 h attachment period, if required, cells were irradiated, incubated at various temperatures for various periods of time in the presence or absence of the indicated inhibitors. Cells were then incubated for 7-14 days, fixed, stained and colonies exceeding 50 cells were counted. NU1025 was added to the cells 24 h before seeding and remained present during the entire duration of the experiment. In experiments involving a combination of NU1025 and 17-DMAG, cells were first treated as indicated and subsequently trypsinized, counted and plated at various concentrations. 17-DMAG was added to the cells 1 h prior to and removed 30-60 min after the incubation at elevated temperature.

### Gene Targeting Efficiency Assay.

The gene targeting assay has been described previously (12)(41). Cells were incubated at elevated temperature from 1 h prior to transfection until 1 or 5 h post transfection. 17-DMAG was added 3 h prior to the hyperthermia treatment and removed 1 h after.

### Irradiation.

Unless stated otherwise, cells were irradiated directly after incubation at elevated temperature, by γ-rays from a cesium source (137Cs, 0.70 Gy/min) or by α-particles as described previously (15). Cells from the cervix carcinoma biopsies were irradiated with α-particles by placing coverslips with cells upside-down on a 1.8 μm-thick polyester membrane and irradiating from below, through the membrane.

### Laser Microirradiation and Time-Lapse Microscopy.

Cells were cultured on glass coverslips for 24 h, then the medium was changed to CO2-independent medium, cells were incubated at 41° C. or 37° C. for 1 h and subsequently placed under a Leica SP2 confocal microscope equipped with heated stage. Cells were then microirradiated with 365 nm line of Innova II argon laser (Coherent) in predefined areas of nuclei. Next, the cells were imaged for required period of time. At least 10 cells were analyzed for each condition tested.

### Image Acquisition and Processing.

Unless stated otherwise, 3-D images of immunofluorescently stained cells were acquired using DMRA fluorescence microscope (Leica) with cooled CCD camera (Apogee), deconvolved using Huygens software (SVI) and processed using Photoshop (Adobe Systems). Wide-field images represent maximum intensity projections of the respective 3-D images, while images and movies acquired using confocal microscope represent one confocal slice. Accumulation of GFP-RAD51 and EGFP-KU80 at the areas exposed to UVA light was analyzed by measuring the mean intensity of the GFP signal in the selected areas using ImageJ.

### Experimental Setup and Results.

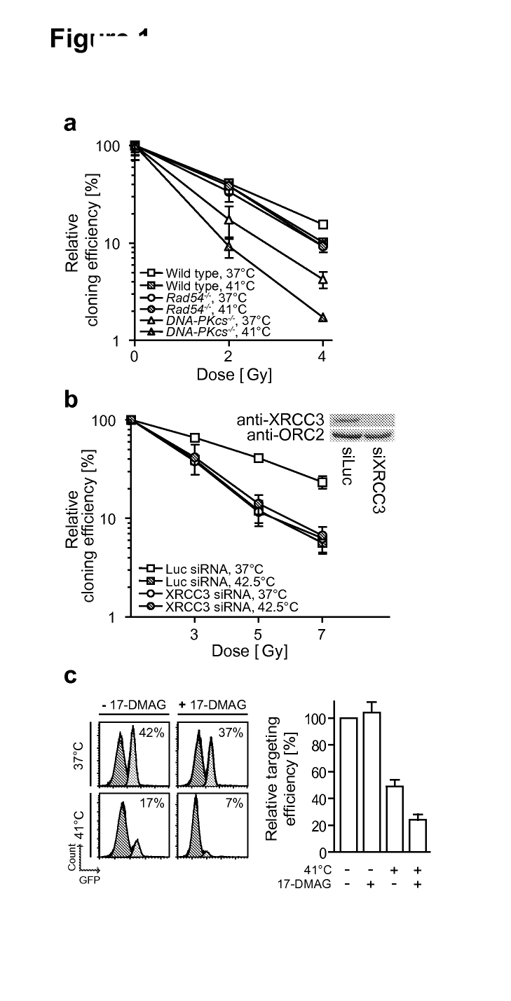

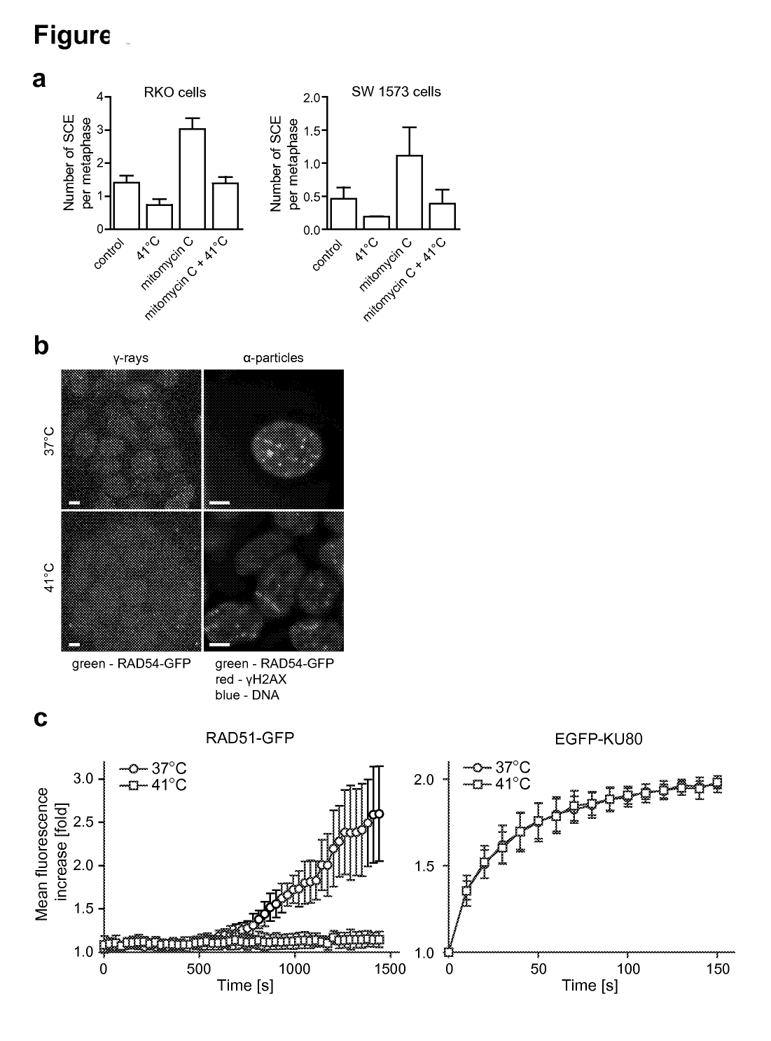

To determine target(s) of mild hyperthermia in mammalian DSB repair pathways, we measured radiosensitization of wild-type, DNA-PKCS−/− and Rad54−/− mouse embryonic stem (ES) cells incubated at 41° C. for 75 min. We chose this temperature for two reasons. First, higher temperatures (>43° C.) might affect a broader range of metabolic activities and thus hamper explicit interpretation of results. Second, temperatures below 43° C. are relevant in clinical practice. Interestingly, we observed that both wild-type and DNA-PKCS−/−, but not Rad54−/− cells were sensitized by hyperthermia (FIG. 1A). The inability to further radiosensitize HR deficient ES cells using hyperthermia was not limited to RAD54 disruption or to mouse ES cells as HeLa cells, in which the HR factor XRCC3 was down-regulated using siRNA, were also refractory to radiosensitization by hyperthermia (FIG. 1B). These results suggest that mild hyperthermia sensitizes cells to ionizing radiation by targeting HR. To directly measure the effect of mild hyperthermia on HR, we determined the efficiency of HR-mediated gene targeting in ES cells. In line with the results from the experiments described above, mild hyperthermia significantly reduced the efficiency of HR-mediated gene targeting (FIG. 1C). Mild hyperthermia also reduced the frequency of sister chromatid exchanges (FIG. 5A) which are to a large extent mediated by HR.

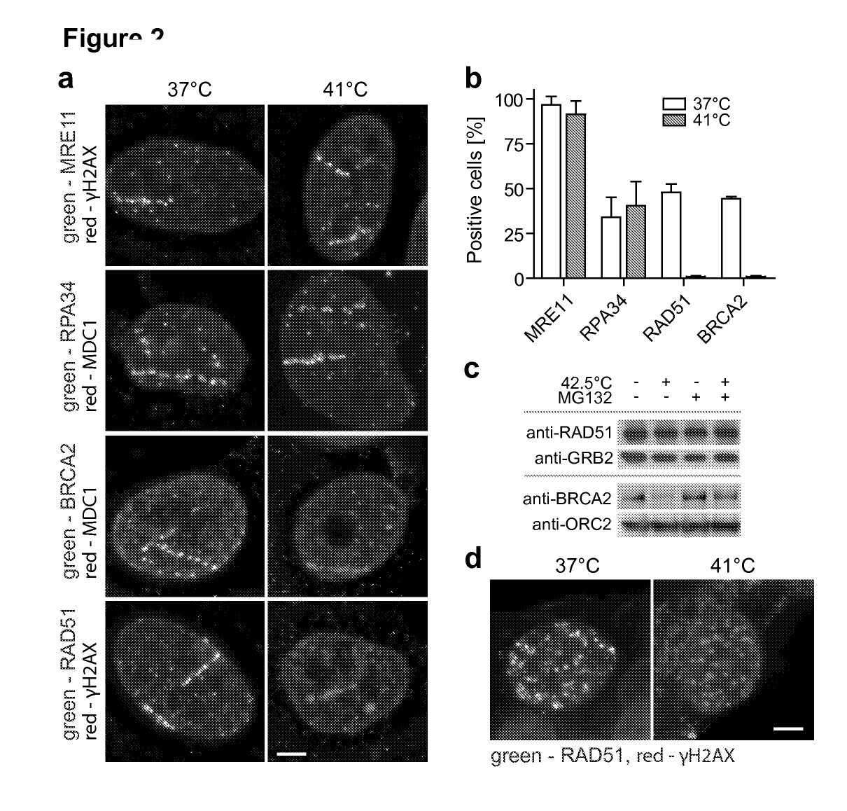

Majority of important DSB repair-related proteins accumulates at sites of radiation-induced DSBs to form IRIF. This accumulation is crucial for proper functioning of repair mechanisms and disturbed formation of IRIF by DSB repair proteins is associated with repair deficiencies and genome instability. To pinpoint the defect(s) in the HR pathway caused by hyperthermia, we examined formation of IRIF by a range of DSB repair factors at alpha-particle induced DSBs in U2OS cells incubated at 37° C. or 41° C. for 60 min prior to irradiation (FIG. 2A, 2B and FIG. 5). Early in HR, the ends of DSBs are resected in a reaction involving the MRE11/RAD50/NBS1, CtIP and BRCA1 complexes generating single-stranded DNA stretches, which are subsequently coated by RPA. MRE11, BRCA1 and RPA efficiently accumulated at DSB sites, regardless of whether the cells had been preincubated at 41° C., suggesting that DSB end resection is unaffected by hyperthermia. In subsequent steps of HR, the RAD51 recombinase forms nucleoprotein filaments on single-stranded DNA with help of BRCA2. While RAD51 and BRCA2 IRIF could be detected 15 min post-irradiation in control cells, preincubation at 41° C. completely abrogated accumulation of both proteins at DSB sites (FIGS. 2A and 2B). Similarly, hyperthermia prevented formation of RAD51 IRIF at alpha-particle induced DSBs in HeLa, SW-1573 and RKO cells (data not shown) as well as of RAD54-GFP IRIF at alpha-particle and X-ray induced DSBs in mouse ES cells (FIG. 5B). Moreover, preincubation at 41° C. abrogated accumulation of GFP-RAD51 in V79 cells, but not EGFP-KU80 in XR-V15B cells at laser-induced DNA damage (FIG. 5C). These results suggest that hyperthermia inhibits the formation of RAD51 nucleoprotein filaments, a pivotal step of HR. Resection of DNA ends, resulting in the single-stranded DNA/RPA filament required by RAD51 appears to be intact, suggesting that hyperthermia could induce defects in RAD51 itself or in BRCA2, which is essential for the loading of RAD51 on the proper DNA substrate and the accumulation of RAD51 in IRIF.

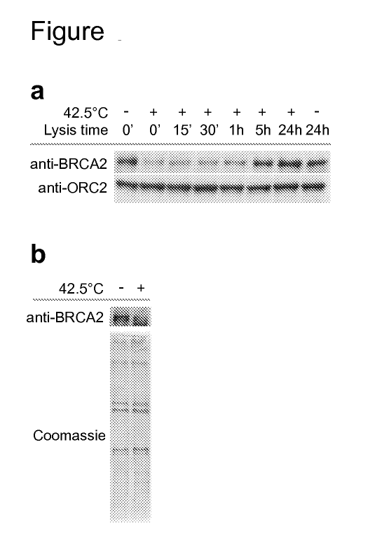

To establish how these two proteins are affected by mild hyperthermia, we analyzed cell extracts by immunoblotting. Preincubation of HeLa cells at elevated temperature had no detectable effect on levels of RAD51, but it did result in considerable reduction of BRCA2 levels (FIG. 2C). The latter effect was also observed in BLM cells (FIG. 6A). This reduction can be explained by proteasome-mediated degradation, as we detected no decrease of BRCA2 levels when cells were exposed to 41° C. in the presence of proteasome inhibitor MG132 (FIG. 2C). Importantly, hyperthermia-induced effects on HR proteins were not limited to cultured cells. They were also observed in cells from fresh tumour biopsies and from normal human skin. Mild hyperthermia eliminated accumulation of RAD51 on alpha-particle induced DSBs in biopsies from a cervix carcinoma (FIG. 2D). Additionally, reduced levels of BRCA2 were detected by immunoblotting in cells from the part of a skin biopsy incubated at 41° C., (FIG. 6B). Taken together, these results indicate that mild hyperthermia might lead to inactivation and degradation of BRCA2 by the proteasome system, resulting in malfunction of HR in human cells, tissues and tumours.

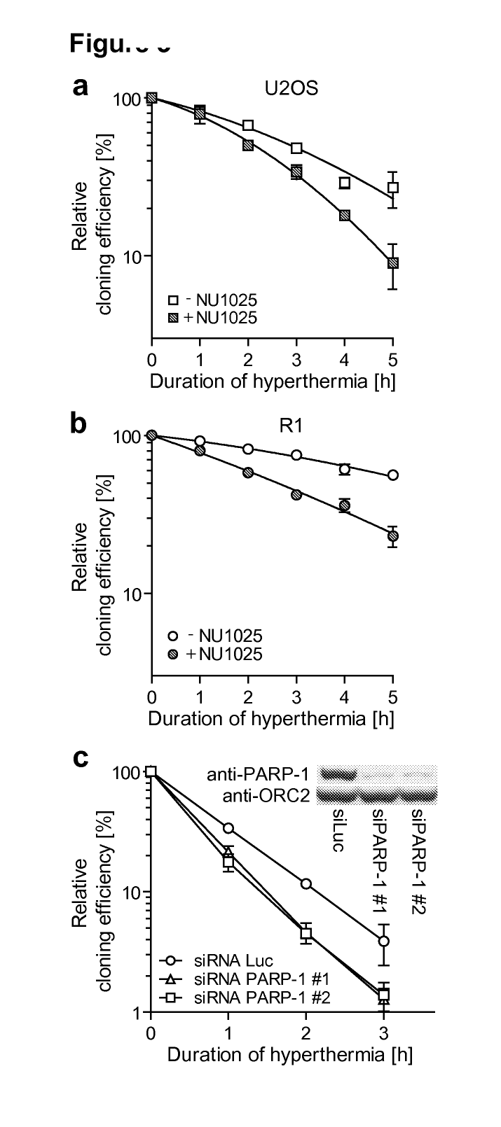

HR-deficient cell lines and tumours, particularly those defective in BRCA2, are extremely sensitive to PARP-1 inhibitors. Therefore, we hypothesized that inhibition of HR by mild hyperthermia would increase the cytotoxicity of PARP-1 inhibitors in HR-proficient cells. As expected, we observed a decrease in clonogenic survival of human U2OS (FIG. 3A) and rat R1 (FIG. 3B) cells incubated at 41° C. in the presence of the PARP-1 inhibitor NU1025, as compared to cells subjected to hyperthermia alone. This effect was proportional to the duration of the heat treatment and correlated with sensitivity of these cells to treatment with hyperthermia or NU1025 alone (data not shown). NU1025 did not reduce survival of hyperthermia-treated SW-1573 or MOS cells, which were also insensitive to either hyperthermia or NU1025 alone (data not shown). Consistently, large variations in the cytotoxicity induced by a number of PARP-1 inhibitors in HR-deficient cells have been reported. To unambiguously demonstrate that reduced PARP-1 activity sensitizes cells to hyperthermia, we analyzed cytotoxic effects of hyperthermia on HeLa cells in which PARP-1 was down-regulated by siRNA. Incubation at elevated temperature resulted in diminished survival of these cells, in line with the results obtained with U2OS and R1 lines (FIG. 3C).

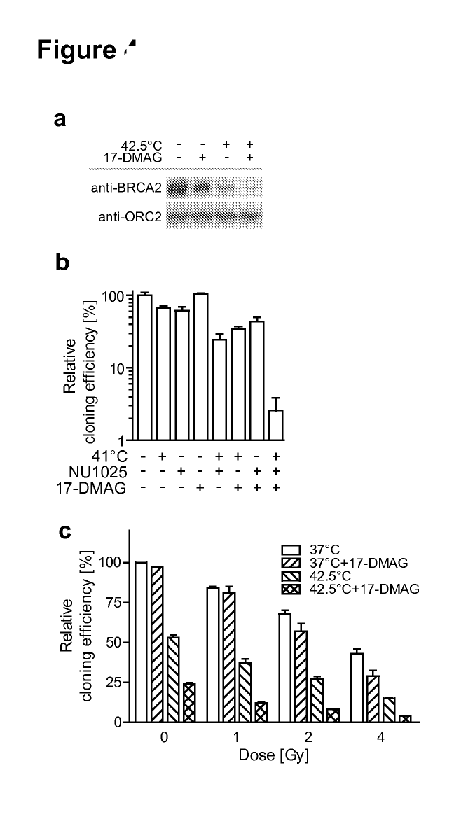

It was found that levels of BRCA2 protein (FIG. 4A) and the efficiency of HR-mediated gene targeting (FIG. 1C) were further reduced in cells treated with hyperthermia in the presence of 17-DMAG, as compared to those heated in the absence of 17-DMAG or incubated with 17-DMAG at 37° C. These results show that BRCA2 is highly prone to degradation in cells exposed to mild hyperthermia in the absence of the protective activities of Hsp90. As a consequence, inhibition of Hsp90 should also enhance the cytotoxic effects of PARP-1 inhibition in hyperthermia-treated cells. Indeed, the clonogenic survival of rat R1 cells incubated at 41° C. was dramatically reduced when they were treated in the presence of NU1025 and 17-DMAG (FIG. 4B). Moreover, our results suggest that by inhibiting HR, the combination of 17-DMAG and hyperthermia should sensitize cells to ionizing radiation exposure. As anticipated, mild hyperthermia applied in the presence of 17-DMAG increased sensitivity of HeLa cells to radiation by 7- to 10-fold, as compared to radiation alone, and by 3- to 4-fold, as compared to hyperthermia treatment alone. (FIG. 4C).

The results presented here indicate that clinically-relevant, mild hyperthermia targets HR by inducing degradation of BRCA2, a key HR factor. We cannot rule out that higher temperatures or prolonged treatments may affect other components of DSB repair pathways. This could explain reports revealing that heat can radiosensitize cells harboring defects in NHEJ or in both repair pathways. We show that impairment of HR by hyperthermia can be enhanced significantly by inhibition of Hsp90. Importantly, our results suggest that heat exposure can be used to (locally) introduce BRCA2 deficiency and thereby render HR-proficient tumour cells highly sensitive to PARP-1 inhibitors. Because our approach is not based on genetic deficiency, it is not likely to exert selective pressure, which might lead to development of resistance. Additionally, we demonstrate that hyperthermia in combination with Hsp90 inhibition efficiently sensitizes cells to ionizing radiation. These results provide a rational basis for development of therapies exploiting (local) induction of HR deficiency in combination with PARP-1 inhibitors, ionizing radiation or other DSB-inducing chemotherapeutic agents.

### Example 2

### In Vivo Tumour Load Reduction by a Combination of Hyperthermia, AZD2281 (PARP-1 Inhibitor) and 17-DMAG (HSP90 Inhibitor)

Tissue culture results are extended to in vivo situations by performing experiments using the syngenic rhabdomyosarcoma rat model (Van Bree et al., Int J Hyperthermia, 1999).

An amount of 3×10⁶ rhabdomyosarcoma cells are injected subcutaneously into both flanks of mature WAG/Rij rats. Within 3 weeks, the animals develop tumours of 1500 mm³, at which point the tumours are surgically removed, cut into fragments of 1 mm3, and single fragment are implanted into one or both hind legs of adult rats. The animals are divided into groups (see below). After 3 weeks, the implanted tumours reach ˜200 mm³. At this point, the animals receive different treatments for 4 weeks at intervals of 3 days as indicated below:

Group 1: Sham hyperthermia treatment (HT) (n=4, 2 tumors per animal)

Group 2: HT only (90 min incubation in a waterbath set at 42° C., n=4, tumors in both hind legs)

Group 3: PARP-1 inhibitor AZD2281 only (n=4, tumors in both hind legs)

Group 4: HSP-90 inhibitor 17DMAG only (n=4, tumors in both hind legs)

Group 5: 17DMAG+AZD2281 (n=4, tumors in both hind legs)

Group 6: HT+AZD2281 (n=12, 1 tumor in 1 hind leg)

Group 7: HT+17DMAG (n=12, 1 tumor in 1 hind leg)

Group 8: HT+17DMAG +AZD2281 (n=12, 1 tumor in 1 hind leg)

AZD2281 (50 mg/kg) is dissolved in 10% DMSO/9% HPBCD-PBS and administered P.O.

17-DMAG (10 mg/kg) is dissolved in physiologic salt and administered I.P.

Inhibitors or solvents (as required by the group specifications above) are administered 24 h and 1 h before the hyperthermia treatment or sham hyperthermia treatment (as required).

The changes of the tumour volumes are monitored using a caliper.

Tumour volumes are normalized to the volumes measured on the day of the first treatment.

### Example 3

### In Vivo Tumour Load Reduction by a Combination of Hyperthermia, AZD2281 and/or PJ-34 (PARP-1 Inhibitors) and 17-DMAG (HSP90 Inhibitor)

Tissue culture results are extended to in vivo situations by performing experiments using the B16BL6 melanoma tumor model (Hart I. R. Am J Pathol. 1979. 97(3):587-600; Ten Hagen T. L. and Eggermont A. M. Int J Hyperthermia. 2008. 24(3):291-9). Tumors are grown as xenografts in the flanks of nude mice. Animals are injected subcutaneously in both sides with 10⁶ B16BL6 cells. Within 3 weeks, the animals develop tumours of approximately 300 mm³, sufficient to yield tumor tissue for implantation of tumor sections of about 5×10⁶ cells subcutaneously in the hind leg of mice. After 3 weeks, this strategy results in ˜75% of tumor-take and tumor dimensions of ˜200 mm³.

Animal groups bearing tumors are then injected intraperitoneally with various combinations of vehicle, AZD2281 and/or PJ-34 (PARP-1 inhibitors) and 17-DMAG (HSP inhibitor). Initial doses are 1, 5, 10 mg/kg for PJ-34, 50, 100 mg/kg for AZD2281 and 5 and 10 mg/kg for 17-DMAG. The tumors are then exposed to hyperthermia by submersion in a water bath at a sufficient temperature to achieve a tumor temperature of 41° C. During the course of the application of hyperthermia, the mice are cooled to maintain body temperatures at or below 41° C. Body temperature is monitored rectally using a thermocouple. The treatments are performed once or repeated twice a week for 2 weeks. The tumor volume, calculated from 3 maximal dimensions according to the formula V=n*x*y*z/6, is measured twice a week using a Vernier caliper. Tumour volumes are normalized to the volumes measured on the day of the first treatment.

1. A method of treating or preventing hyperproliferative disease in a body tissue of a subject, comprising the steps of:
a) administering to a subject in need thereof a therapeutically effective amount of an agent that induces double strand breaks in the DNA (DSB-inducing agent) of the hyperproliferative cells of said body tissue; and
b) subjecting the hyperproliferative cells of said body tissue to hyperthermia prior to, simultaneously with or subsequently to step a) to thereby induce in said cells the degradation, inhibition and/or inactivation of BRCA2, and
wherein said method further comprises administering to said subject a therapeutically effective amount of a heat shock protein inhibitor.

2. Method according to claim 1, wherein the degradation, inhibition and/or inactivation of BRCA2 in said cells is essentially complete or wherein the degradation, inhibition and/or inactivation of BRCA2 in said cells is at least to the extent that cells are rendered sensitive to DSB-inducing agents.

3. Method according to claim 1, wherein said hyperproliferative disease is cancer.

4. Method according to claim 1, wherein said DSB-inducing agent is a PARP-inhibitor.

5. Method according to claim 4, wherein said PARP-inhibitor is selected from the group consisting of 5-aminoisoquinolinone; 3-methyl-5-aminoisoquinolinone; 3-aminobenzamide; 5-iodo-6-amino-1,2-benzopyrone; 3,4-dihydro-5[4-(1-piperindinyl)butoxy]-1(2H)-isoquinoline; 1,5-dihydroxyisoquinoline; -aza-5[H]-phenanthridin-6-ones; 6(5H)-phenanthridinone; 4-amino-1,8-naphthalimide; 8-hydroxy-2-methylquinazoline-4-one; N-(6-oxo-5,6-dihydrophenanthridin-2-yl)-N,N-dimethylacetamide; indeno-isoquinolinone; 5-chloro-2-[3-(4-phenyl-3,6-dihydro-1(2H)-pyridinyl)propyl]-4(3H)-quinazolinone; 1-piperazineacetamide,4-[1-(6-amino-9H-purin-9-yl)-1-deoxy-β-D-ribofuranuron]-N-(2,3-dihydro-1H-isoindol-4-yl)-1-one; thieno[2,3-c]isoquinolin-5-one; 2-dimethylaminomethyl-4H-thieno [2,3-c]isoquinolin-5-one; 4-hydroxyquinazoline; nicotinamide; minocycline; 2-methyl-3,5,7,8-tetrahydrothiopyrano[4,3-d]pyrimidine-4-one; 3-(4-chlorophenyl)quinoxaline-5-carboxamide; benzamide; N-(6-oxo-5,6-dihydrophenanthridin-2-yl)-2-(N,N-dimethylamino)acetamide; AG014699; AG14361; 2-[(R)-2-methylpyrrolidin-2-yl]-1H-benzimidazole-4-carboxamide; 4-[3-(4-cyclopropanecarbonylpiperazine-1-carbonyl)-4-fluorobenzyl]-2H-phthalazin-1-one; BSI-401; BSI-201; CEP-8983; CEP-9722;
GPI-21016; GPI 16346; GPI 18180; GPI 6150; GPI 18078; GPI 6000; 2-aminothiazole analogues; quinoline-8-carboxamides; 2- and 3-substituted quinoline-8-carboxamides; 2-methylquinoline-8-carboxamide; 2-(1-propylpiperidin-4-yl)-1H-benzimidazole-4-carboxamide; aminoethyl pyrrolo dihydroisoquinolinones; imidazoquinolinone and derivatives thereof; imidazopyridine and derivatives thereof; isoquinolindione and derivatives thereof; 2-[4-(5-Methyl-1H-imidazol-4-yl)-piperidin-1-yl]-4,5-dihydro-imidazo[4,5,1-i,j]quinolin-6-one; 2-(4-pyridin-2-yl-phenyl)-4,5-dihydro-imidazo[4,5,1-i,j]quinolin-6-one; 6-chloro-8-hydroxy-2,3-dimethyl-imidazo-[1,2-α]-pyridine; 4-(1-methyl-1H-pyrrol-2-ylmethylene)-4H-isoquinolin-1,3-dione; E7016; 2-[methoxycarbonyl(4-methoxyphenyl)methylsulfanyl]-1H-benzimidazole-4-carboxylic Acid Amide; 4-carboxamidobenzimidazole-2-ylpyrroline; tetrahydropyridine nitroxides derivatives; N-[3-(4-oxo-3,4-dihydro-phthalazin-1-yl)phenyl]-4-(morpholin-4-yl) butanamide methanesulfonate monohydratate; phenanthridinone; 4-iodo-3-nitrobenzamide; 2-(4-hydroxyphenyl)-1H-benzimidazole-4-carboxamide; 2-aryl-1H-benzimidazole-4-carboxamides; 2-phenyl benzimidazole 4-carboxamides; phthalazin-1(2H)-one; 3-substituted 4-benzyl-2H-phthalazin-1-ones and derivatives; combinations of the above, analogues and derivatives and pharmaceutically acceptable salts thereof.

6. Method according to claim 1, wherein said subjection to hyperthermia involves a temperature of between 41.0 and 45.0° C.

7. Method according to claim 1, wherein said effective amount of DSB-inducing agent is about 0.0001 mg to about 1000 mg per kg body weight of said subject per 24 hours.

8. (canceled)

9. Method according to claim 1, wherein said heat shock protein inhibitor is a HSP 90 inhibitor.

10. Method according to claim 9, wherein said therapeutically effective amount of said heat shock protein inhibitor is about 0.0001 mg to about 1000 mg per kg body weight of said subject per 24 hours.

11. A method of killing cells, comprising (a) administering to the cells a cytotoxic amount of a PARP-inhibitor, and (b) subjecting the cells to hyperthermia prior to, simultaneously with, or subsequently to step a), so as to inactivate homologous recombination (HR) in said cells, and said method further comprising exposing said cells to a therapeutically effective amount of an HSP90 inhibitor.

12. A method for inhibiting homologous recombination in cells comprising inducing the degradation, inhibition and/or inactivation of BRCA2 in said cells by hyperthermia, and said method further comprising exposing said cells to a therapeutically effective amount of an HSP90 inhibitor.

13. Method according to claim 12, wherein the degradation, inhibition and/or inactivation of BRCA2 in said cells is essentially complete or wherein the degradation, inhibition and/or inactivation of BRCA2 in said cells is at least to the extent that cells are rendered sensitive to PARP1 or other DSB-inducing agent.

14-15. (canceled)
